SEQUENCE LISTING (3 sequences)

SEQ ID NO: 1 - DNA, 15 bp, Artificial sequence
  Synthetic probe
  misc_feature (1)..(10): n is a, c, g, or t
nnnnnnnnnnttttt

SEQ ID NO: 2 - DNA, 10 bp, Artificial sequence
  Synthetic probe
  misc_feature (1)..(10): n is a, c, g, or t
nnnnnnnnnn

SEQ ID NO: 3 - DNA, 10 bp, Artificial sequence
  Synthetic probe
  misc_feature (1)..(5): n is a, c, g, or t
nnnnnttttt

(19) United States
(12) Patent Application Publication
(10) Pub. No.: US 2023/0120072 A1
(43) Pub. Date: Apr. 20, 2023

(54) METHODS FOR EXTRACTING AND SEQUENCING SINGLE-STRANDED DNA AND RNA FROM NON-TREATED BIOSPECIMENS

(71) Applicant: William Marsh Rice University, Houston, TX (US)

(72) Inventors: David ZHANG, Houston, TX (US); Ruojia WU, Houston, TX (US); Peng DAI, Houston, TX (US); Yuxuan CHENG, Houston, TX (US); Xiangjiang WANG, Houston, TX (US)

(73) Assignee: William Marsh Rice University, Houston, TX (US)

(21) Appl. No.: 17/787,290

(22) PCT Filed: Dec. 18, 2020
(86) PCT No.: PCT/US20/66152

§ 371 Date: Jun. 17, 2022

(51) Int. Cl. C12Q 1/6874

(52) U.S. Cl. CPC C12Q 1/6874; C12Q 1/6806

(57) ABSTRACT

Provided herein are hybrid capture-based methods to extract single-stranded DNA or RNA directly from non-treated biospecimens. The methods allow for the detection and analysis of unexplored short single-stranded DNA (sssDNA, mean length 50 nt) and ultrashort single-stranded DNA (ussDNA, mean length 15 nt) of human origin present in the biospecimen. The methods allow the discovery of unexplored short single-stranded DNA (sssDNA) in isolated red blood cells, which were believed to be deprived of nucleic acids because of the lack of a nucleus in mature red blood cells. The DNA or RNA extracted using the disclosed methods can be used as disease prognostic biomarkers and treatment predictive biomarkers.

### REFERENCE TO RELATED APPLICATIONS

The present application claims the priority benefit of U.S. provisional application No. 62/951,069, filed Dec. 20, 2019, the entire contents of which is incorporated herein by reference.

### STATEMENT REGARDING FEDERALLY SPONSORED RESEARCH

This invention was made with government support under Grant No. R01 HG008752 awarded by the National Institutes of Health. The government has certain rights in the invention.

### BACKGROUND

The development of this disclosure was funded in part by the Cancer Prevention and Research Institute of Texas (CPRIT) under Grant No. RP180147.

### 1. Field

The present invention relates generally to the field of molecular biology. More particularly, it concerns methods for detecting and analyzing short single-stranded DNA, ultrashort single-stranded DNA and RNA in various biospecimens, and in particular in non-treated biospecimens.

### 2. Description of Related Art

Nucleic acid has emerged as an important analyte in molecular testing due to the richness of information in even minimal amount of material. Cellular genomic DNA or RNA is widely used in oncology, forensics, paternity testing, and research. Precision medicine relies on genomic information to provide guidance for individualized therapies, including diagnosis and prognosis for a variety of diseases including cancer, neurodegenerative diseases, and infectious diseases. The discovery of new classes of DNA biomarkers has preceded significant advances in diagnostics and benefited human health. The first wave of precision medicine was based on the analysis of germline mutations and SNPs from leukocyte or buccal swab samples to inform disease risk and drug dosage. Subsequently, nucleic acid biomarkers were expanded to include RNA expression patterns, DNA mutations in tumor tissue samples, circulating tumor cells (CTCs), cell-free DNA (cfDNA) and exosome-derived DNA from peripheral blood plasma. The classes, lengths, and sources of nucleic acid biomarkers are summarized in FIG. 1A.

One class of DNA biomarkers currently evaluated to have high translational value is cfDNA, double-stranded DNA in peripheral blood plasma with length around 165 base pairs (bp). Because cfDNA molecules are released through cell death or active secretion and are quickly cleared from the bloodstream with a half-life between 5 and 150 min, they capture a “snapshot” of dying cells throughput the whole body. Cell-free DNA have had transformative impact on both non-invasive prenatal testing (NIPT), organ transplant rejection monitoring, and cancer therapy selection and remission monitoring. Other examples of nucleic acid biomarkers being extensively studies are micro RNAs (miRNAs), long non-coding RNA, and exosome-derived DNA and RNA.

Despite their active footprint in both translational medicine and research, the current methods for purification of nucleic acids, including circulating DNA from plasma, systematically excludes the purification of other nucleic acid biomarkers. The most commonly used methods in commercial products based on silica-DNA interactions, based on columns or beads (e.g. QIAamp circulating nucleic acid kit (Qiagen), Cobas cfDNA sample preparation kit (Roche), or Apostle Minimax High Efficiency Cell-Free DNA Isolation Kit (Beckman Coulter)) systematically fail to extract DNA shorter than about 50 nt because those DNA molecules fail to bind to the columns or beads (FIGS. 1B-C). Furthermore, DNA molecules are believed to be double stranded by default, and the downstream preparation (e.g., double strand ligation) based on the double-strandedness assumption fail to analyze any single stranded DNA molecules.

### SUMMARY

Provided herein are DNA extraction methods that are suitable for capturing single-stranded nucleic acid molecules, or nucleic acid molecules with partially single-stranded domains from un-treated biospecimens. The capture methods only involve mixing and incubating biospecimens with probes and hybrid capture buffers. In some embodiments, the captured molecules are analyzed by next generation sequencing with amendments of appropriate sequencing adapters. With the direct capture from biospecimen (DCB) approach, human red blood cells (RBCs) were found to be highly enriched in short single-stranded DNA (sssDNA), which is the opposite of expectation because RBCs have long been believed to be deprived of DNA due to the lack of nuclei in mature RBCs. On the other hand, sssDNA was found to be depleted in human plasma. Furthermore, sssDNAs were also found in biospecimens from non-human species. These findings indicate that the sssDNA might be a distinct DNA type in human and other species existing in cell membrane- or RBC membrane-bound format.

In one embodiment, provided herein are mixtures for direct capture from red blood cells comprising (1) isolated red blood cells that do not contain greater than 1 part in 1000 white blood cells and (2) an oligonucleotide capture probe with length between 5 nt and 100 nt (e.g., between 5 nt and 90 nt, between 5 nt and 80 nt, between 5 nt and 70 nt, between 5 nt and 60 nt, between 5 nt and 50 nt, between 10 and 100 nt, between 10 nt and 90 nt, between 10 nt and 80 nt, between 10 and 70 nt, between 10 and 60 nt, between 10 and 50 nt, or any range derivable therein) comprising (a) degenerate LNA nucleotides at between 2 and 50 loci (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 9, or 50 loci, or any range derivable therein) that do not allow polymerase extension or ligation and that do not include electrochemically active component and (b) an affinity tag modification at 3′, wherein the mixture does not comprise reverse transcriptase. In some aspects, the biospecimen includes but is not limited to red blood cells isolated from venous blood from human or non-human animals. In some aspects, the biospecimen includes but is not limited to red blood cells isolated from arterial blood from human or non-human animals. In some aspects, the red blood cell samples are not subjected to (1) storage at temperature above 4° C. for more than 48 hrs after sample collection; (2) heating above 45° C.; (3) enzymatic treatment (e.g. protease treatment); (4) harsh chemical treatment (e.g. lysis treatment); and/or (5) harsh physical treatment including but is not limited to shearing, electroporation, sonication. In some aspects, the affinity tag in capture probe includes but is not limited to (1) noncovalent affinity tags such as biotin, and (2) covalent affinity tags (reaction handle) such as azide, alkyne functional groups. In some aspects, the oligonucleotide of the capture probe comprises one or more non-natural degenerate bases with non-natural backbone modifications, such as locked nucleic acids. In some aspects, the oligonucleotide of the capture probe comprises one or more non-natural degenerate bases with universal affinity, such as inosine or 5-nitroindole. In some aspects, the hybrid capture buffer comprises (1) cation with concentration greater than 1 mM, (2) tween 20 with volume concentration between 0.01% and 1%, (3) Tris with concentration between 1 mM to 100 mM, (4) ethylenediaminetetraacetic acid (EDTA) with concentration between 1 mM to 100 mM, (5) sodium dodecyl sulfate (SDS) with volume concentration between 0.01% and 1%, and/or (6) tetramethylammonium chloride (TMAC) with concentration between 0 and 3 M.

In one embodiment, provided herein are methods for capturing sssDNA from red blood cells (RBCs), the methods comprising (1) isolating RBCs from freshly drawn blood; (2) mixing isolated RBCs with a capture probe comprising oligonucleotide with length between 5 nt and 100 nt (e.g., between 5 nt and 90 nt, between 5 nt and 80 nt, between 5 nt and 70 nt, between 5 nt and 60 nt, between 5 nt and 50 nt, between 10 and 100 nt, between 10 nt and 90 nt, between 10 nt and 80 nt, between 10 and 70 nt, between 10 and 60 nt, between 10 and 50 nt, or any range derivable therein) and an affinity tag and a buffer; (3) incubating the mixture from (2) at temperature between 0° C. and 45° C. (e.g., 0, 1, 2, 3, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45° C., or any range derivable therein) for between 1 second and 1 day (e.g., 1 second, 30 seconds, 1 minute, 2 minutes, 5 minutes, 30 minutes, 1 hour, 2 hours, 6 hours, 12 hours, 18 hours, or 24 hours, or any range derivable therein) to allow for hybridization between sssDNA and capture probe; (4) collecting the capture probes using the affinity tag; and (5) washing the collected capture probes to remove unbound substances and collecting captured DNA in elution buffer.

In some aspects, freshly drawn blood is collected in anticoagulant coated tubes. In some aspects, methods for red blood cell isolation include but are not limited to density gradient centrifugation, fluorescence-activated cell sorting (FACS), and white blood cell depletion using immunomagnetic cell separation. In some aspects, the biospecimens are not subjected to (1) storage at temperature above 4° C. for more than 48 hrs after sample collection; (2) freeze-thaw for total blood samples; (3) heating above 45° C.; (4) enzymatic treatment (e.g. protease treatment); (5) chemical treatment (e.g. lysis treatment); and/or (6) harsh physical treatment including but is not limited to shearing, electroporation, sonication.

In some aspects, the affinity tag in capture probe includes but is not limited to (1) noncovalent affinity tags such as biotin, and (2) covalent affinity tags (reaction handle) such as azide or alkyne functional groups. In some aspects, the oligonucleotide of the capture probe comprises unmodified degenerate base stretch between 5 nt and 100 nt (e.g., between 5 nt and 90 nt, between 5 nt and 80 nt, between 5 nt and 70 nt, between 5 nt and 60 nt, between 5 nt and 50 nt, between 10 and 100 nt, between 10 nt and 90 nt, between 10 nt and 80 nt, between 10 and 70 nt, between 10 and 60 nt, between 10 and 50 nt, or any range derivable therein). In some aspects, the oligonucleotide of the capture probe comprises DNA oligonucleotide between 5 nt and 100 nt (e.g., between 5 nt and 90 nt, between 5 nt and 80 nt, between 5 nt and 70 nt, between 5 nt and 60 nt, between 5 nt and 50 nt, between 10 and 100 nt, between 10 nt and 90 nt, between 10 nt and 80 nt, between 10 and 70 nt, between 10 and 60 nt, between 10 and 50 nt, or any range derivable therein). In some aspects, the oligonucleotide of the capture probe comprises one or more non-natural degenerate bases with non-natural backbone modifications, such as locked nucleic acids. In some aspects, the oligonucleotide of the capture probe comprises one or more non-natural degenerate bases with universal affinity, such as inosine or 5-nitroindole. In some aspects, the concentration of the capture probe is between 50 pM and 5 μM (e.g., 50 pM, 100 pM, 500 pM, 1 nM, 50 nM, 100 nM, 500 nM, 1 μM, or 5 μM, or any range derivable therein).

In some aspects, the hybrid capture buffer comprises (1) cation with concentration greater than 1 mM; (2) tween 20 with volume concentration between 0.01% and 1%; (3) Tris with concentration between 1 mM to 100 mM; (4) ethylenediaminetetraacetic acid (EDTA) with concentration between 1 mM to 100 mM; (5) sodium dodecyl sulfate (SDS) with volume concentration between 0.01% and 1%; and/or (6) tetramethylammonium chloride (TMAC) with concentration between 0 and 3 M.

In some aspects, the method comprises RNase treatment to retain only one species of nucleic acids.

In some aspects, the method comprises using ligation and/or PCR approaches to append terminal sequences at 5′ and/or 3′ of single-stranded nucleic acid molecules. The appended terminal sequences can be adapter and index sequences for high-throughput sequencing. In some aspects, the method comprises amplifying the index-appended single-stranded molecules with index primers to increase concentration. In some aspects, the high-throughput sequencing is performed via sequencing-by-synthesis. In some aspects, the high-throughput sequencing is performed via sequence-specific current measurements in conjunction with nanopores.

In one embodiment, provided herein are methods for using sssDNA as disease prognostic biomarkers and treatment predictive biomarkers, based on mutation sequence variance in sssDNA. In some aspect, the sssDNAs are extracted and prepared for sequencing via methods described herein. In some aspects, sssDNAs can be prepared for methylation analysis, wherein extracted sssDNAs treated with bisulfite conversion reagents to transform all unmethylated cytosine to uracil prior to library preparation for high-throughput sequencing. In some aspects, sssDNAs can be prepared for methylation analysis, wherein extracted sssDNAs treated with oxidization reagents (e.g. TET2) and APOBEC to transform all unmethylated cytosine to uracil prior to library preparation for high-throughput sequencing. In some aspects, the lengths of sssDNAs are analyzed from high-throughput sequencing data, and if the sssDNAs are longer than sequencing read length, their lengths are inferred from aligned genomic positions of pair-end reads. In some aspects, genetic alterations, including but are not limited to single nucleotide variation, deletion, insertion, translocation and inversion, are analyzed to evaluate their association with disease and disease status. In some aspects, epigenetic alterations, most likely methylation patterns, are analyzed to evaluate their association with disease and disease status. In some aspects, expression profiles, including but are not limited to point mutations, fusion mutations, and expression levels, are analyzed to evaluate their association with disease and disease status.

In one embodiment, provided herein are methods for using sssDNA as disease prognostic biomarkers and treatment predictive biomarkers, based on quantitative relative concentration of sssDNA at different genome loci. In some aspects, the sssDNAs are extracted and prepared for sequencing via methods described herein. In some aspects, the lengths of sssDNAs are analyzed from high-throughput sequencing data, and if the sssDNAs are longer than sequencing read length, their lengths are inferred from aligned genomic positions of pair-end reads. In some aspects, the total concentrations of sssDNAs in biospecimens or in different compartment of biospecimens are estimated via spiking-in of synthetic reference sssDNA strands. In some aspects, sssDNAs aligned to different genomic loci are normalized to those aligned to reference loci (e.g., housekeeping genes, Alu sequences) to estimate relative concentrations at different genomic loci. In some aspects, the genomic loci of interest include but is not limited to promoter regions, 5′- and 3-′ UTRs, oncogenes, tumor suppressor genes, genes regulating immune responses or neurological activities. In some aspects, metagenomics of sssDNAs is analyzed for DNA concentrations of different bacteria populations. In some aspects, captured sssDNAs are analyzed for aneuploidy related to non-invasive prenatal testing (NIPT) or cancer copy number variation.

In one embodiment, provided herein are methods for the direct capture and extraction of single-stranded DNA (ssDNA) from a biospecimen, the methods comprising: (a) incubating a non-treated biospecimen with a DNA probe comprising an affinity tag and an oligonucleotide at a temperature between 0° C. and 45° C. (e.g., 0, 1, 2, 3, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45° C., or any range derivable therein) in a solution comprising between 0.05 molar and 6 molar monovalent cations, or comprising between 0.001 molar and 2 molar divalent cations, or comprising both between 0.05 molar and 6 molar monovalent cations and between 0.001 molar and 2 molar divalent cations, for between 1 second and 1 day (e.g., 1 second, 30 seconds, 1 minute, 2 minutes, 5 minutes, 30 minutes, 1 hour, 2 hours, 6 hours, 12 hours, 18 hours, or 24 hours, or any range derivable therein) to allow for hybridization between the DNA probe and ssDNA in the biospecimen; (b) collecting the DNA probes using the affinity tag; and (c) washing the collected DNA probes to remove any non-hybridized contaminates from the biospecimen.

In one embodiment, provided herein are methods for direct capture and extraction of RNA from a biospecimen, the methods comprising: (a) incubating a non-treated biospecimen with an RNase inhibitor and a DNA probe comprising an affinity tag and an oligonucleotide at a temperature between 0° C. and 45° C. (e.g., 0, 1, 2, 3, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45° C., or any range derivable therein)in a solution comprising between 0.05 molar and 6 molar monovalent cations, or comprising between 0.001 molar and 2 molar divalent cations, or comprising both between 0.05 molar and 6 molar monovalent cations and between 0.001 molar and 2 molar divalent cations, for between 1 second and 1 day (e.g., 1 second, 30 seconds, 1 minute, 2 minutes, 5 minutes, 30 minutes, 1 hour, 2 hours, 6 hours, 12 hours, 18 hours, or 24 hours, or any range derivable therein) to allow for hybridization between the DNA probe and RNA in the biospecimen; (b) collecting the DNA probes using the affinity tag; and (c) washing the collected DNA probes to remove any non-hybridized contaminates from the biospecimen.

In some aspects of any of the above embodiments, the non-treated biospecimen has not been heated above 45° C. prior to performing the method, has not undergone any biological treatments prior to performing the method, has not undergone any enzymatic reactions prior to performing the method, has not been treated with proteinase K prior to performing the method, has not undergone any chemical treatments prior to performing the method, has not undergone any harsh physical treatments prior to performing the method, has not been sheared prior to performing the method, has not been electroporated prior to performing the method, and/or has not been sonicated prior to performing the method.

In one embodiment, provided herein are methods for direct capture and extraction of single-stranded DNA (ssDNA) and double-stranded DNA (dsDNA) from a biospecimen, the method comprising: (a) heating the biospecimen at a minimum of 90° C. for a minimum of 10 seconds to allow for denaturation of dsDNA; (b) contacting the biospecimen with a capture probe comprising an oligonucleotide having a length between 5 nt and 100 nt (e.g., between 5 nt and 90 nt, between 5 nt and 80 nt, between 5 nt and 70 nt, between 5 nt and 60 nt, between 5 nt and 50 nt, between 10 and 100 nt, between 10 nt and 90 nt, between 10 nt and 80 nt, between 10 and 70 nt, between 10 and 60 nt, between 10 and 50 nt, or any range derivable therein) and an affinity tag that allows for strong association with a solid-state substance; (c) incubating the biospecimen with the capture probe at a temperature between 0° C. and 45° C. (e.g., 0, 1, 2, 3, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45° C., or any range derivable therein) for between 1 second and 1 day (e.g., 1 second, 30 seconds, 1 minute, 2 minutes, 5 minutes, 30 minutes, 1 hour, 2 hours, 6 hours, 12 hours, 18 hours, or 24 hours, or any range derivable therein) to allow for hybridization between the capture probe and nucleic acids in the biospecimen; (d) collecting the capture probes using the affinity tag; and (e) washing the collected capture probes to remove any non-hybridized contaminates from the biospecimen and collecting the capture nucleic acid.

In some aspects, the biospecimen comprises isolated red blood cells, isolated platelets, isolated white blood cells, blood, plasma, serum, urine, cerebrospinal fluid, and/or sputum. In some aspects of any of the above embodiments, the biospecimen is selected from the group consisting of plasma, serum, blood, urine, cerebrospinal fluid, and sputum. In some aspects, the biospecimen is from a human, an animal, a plant, or a bacterium. In some aspects, the biospecimen is a human biospecimen, and wherein the extracted ssDNA is human. In some aspects, the biospecimen is a human microbiome specimen. In some aspects, the human microbiome specimen is an oral, a skin, a vaginal, or a fecal biospecimen.

In some aspects, the biospecimen has not undergone any biological treatments prior to performing the method, has not undergone any enzymatic reactions prior to performing the method, has not been treated with proteinase K prior to performing the method, has not undergone any chemical treatments prior to performing the method, has not been lysed prior to performing the method, has not undergone any harsh physical treatments prior to performing the method, has not been sheared prior to performing the method, has not been electroporated prior to performing the method, and/or has not been sonicated prior to performing the method. In some aspects, the biospecimen is treated with a protease prior to step (a). In some aspects, the biospecimen has not been stored at a temperature above 4° C. for more than 48 hours prior to performing the method.

In some aspects of any of the above embodiments, the affinity tag is a noncovalent affinity tag, such as, for example biotin. In some aspects of any of the above embodiments, step (d) is performed via streptavidin-coated magnetic beads and collecting is performed using a magnet. In some aspects of any of the above embodiments, step (d) is performed via streptavidin-coated agarose beads and collecting is performed using centrifugal force. In some aspects of any of the above embodiments, the affinity tag is a covalent affinity tag (e.g., a reaction handle), such as, for example, an azide or alkyne functional group.

In some aspects of any of the above embodiments, the oligonucleotide of the capture probe comprises a region of degenerate bases. The region of degenerate bases may comprise between 5 and 100 degenerate bases (e.g., about 10 degenerate bases; e.g., between 5 and 90 degenerate bases, between 5 and 80 degenerate bases, between 5 and 70 degenerate bases, between 5 and 60 degenerate bases, between 5 and 50 degenerate bases, between 10 and 100 degenerate bases, between 10 and 90 degenerate bases, between 10 and 80 degenerate bases, between 10 and 70 degenerate bases, between 10 and 60 degenerate bases, between 10 and 50 degenerate bases, or any range derivable therein). Each degenerate base position may be any one of A, G, T or C. The region of degenerate bases may be located at the 5′ end of the oligonucleotide. In some aspects of any of the above embodiments, the oligonucleotide may further comprise a region of known bases. The region of known bases may comprise about 5 thymidines. The region of known bases may be located between the region of degenerate bases and the affinity tag.

In some aspects, the oligonucleotide of the capture probe is a DNA oligonucleotide. In some aspects, the oligonucleotide of the capture probe comprises one or more non-natural degenerate bases with non-natural backbone modifications. In some aspects, the oligonucleotide of the capture probe comprises locked nucleic acids. In some aspects, the oligonucleotide of the capture probe comprises one or more non-natural degenerate bases with universal affinity. In some aspects, the non-natural degenerate bases with universal affinity are inosine or 5-nitroindole. In some aspects, the concentration of the capture probe is between 50 pM and 5 μM (e.g., 50 pM, 100 pM, 500 pM, 1 nM, 50 nM, 100 nM, 500 nM, 1 μM, or 5 μM, or any range derivable therein).

In some aspects, step (b) further comprises contacting the biospecimen with a hybrid capture buffer, wherein the hybrid capture buffer comprises 100 mM to 1 M sodium chloride, 0.01% (v/v) to 1% (v/v) Tween20, 1 mM to 100 mM Tris, 1 mM to 100 mM ethylenediaminetetraacetic acid (EDTA), 0.01% (v/v) to 1% (v/v) doium dodecyl sulfate (SDS), and 0 M to 3 M tetramethylammonium chloride (TMAC). In some aspects, the hybrid capture buffer comprises between 0.05 molar and 6 molar monovalent cations, or between 0.001 molar and 2 molar divalent cations, or both between 0.05 molar and 6 molar monovalent cations and between 0.001 molar and 2 molar divalent cations.

In some aspects of any of the above embodiments, the capture probe in step (a) is not conjugated to a solid support. In certain aspects of any of the above embodiments, the methods are performed without an anion exchange medium.

In some aspects of any of the above embodiments, the hybridization in step (a) is direct hybridization between the capture probe and ssDNA or RNA in the biospecimen.

In some aspects, the methods comprise treating the biospecimen with an RNase.

In some aspects of any of the above embodiments, the methods further comprise eluting the hybridized nucleic acid from the capture probe. In some aspects of any of the above embodiments, the methods further comprise preparing an NGS library using the eluted nucleic acid. In some aspects, the methods further comprise using ligation and/or PCR to append terminal sequences on the 5′ and/or 3′ ends of the captured single-stranded nucleic acid molecules. In some aspects, the terminal sequences are adapter and index sequences for high-throughput sequencing. In some aspects, the methods further comprise amplifying the index-appended single-stranded molecules using index primers. In some aspects, during the process of NGS library preparation, the extracted nucleic acid is not amplified in a sequence-specific manner. In some aspects of any of the above embodiments, the methods further comprise performing high-throughput sequencing on the NGS library. In some aspects, the high-throughput sequencing is performed via sequencing-by-synthesis. In some aspects, the high-throughput sequencing is performed via sequence-specific current measurements in conjunction with nanopores. In some aspects of any of the above embodiments, the methods further comprise analyzing the sequences of the nucleic acid to predict disease in or select a treatment for a patient from whom the biospecimen was obtained. In some aspects of any of the above embodiments, the methods further comprise analyzing the relative concentrations of the ssDNA derived from various genomic loci to predict disease in or select a treatment for a patient from whom the biospecimen was obtained.

In some aspects of any of the above embodiments, the biospecimen is a human biospecimen, and the extracted nucleic acid is human. In some aspects of any of the above embodiments, the methods are methods of selectively isolating ssDNA or RNA.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C. Short and ultrashort single-stranded DNA (sssDNA and ussDNA) biomarkers in human blood plasma. (FIG. 1A) Length ranges of DNA biomarkers found in blood. Currently, all well-studied DNA biomarker types are double-stranded and longer than ˜100 nt. Short and single-stranded DNA molecules have been understudied due to technical limitations. (FIG. 1B) Short single-stranded DNA are systematically lost during standard DNA extraction methods. Subsequent NGS library preparation methods further bias against short single-stranded DNA. (FIG. 1C) Illustration (not to scale) of length distribution of currently visible cfDNA and ssDNA in blood plasma. Observed cfDNA length distribution is based on experiments using standard cfDNA library preparation methods on blood plasma from a healthy human volunteer.

FIGS. 2A-B. Mixture for direct capture from red blood cells. (FIG. 2A) Composition of the mixture for direct capture from red blood cells comprise isolated red blood cells with short single-stranded DNA and capture probes. (FIG. 2B) Configurations of the oligonucleotide capture probe. (NNNNNNNNNN=SEQ ID NO: 2; NNNNNTTTTT=SEQ ID NO: 3)

FIGS. 3A-C. Direct capture of sssDNA from red blood cells. (FIG. 3A) Direct capture from RBC workflow. A biotin-modified DNA capture probe bearing degenerate poly-N random sequence (NNNNNNNNNN=SEQ ID NO: 2) is directly mixed with isolated RBC in hybrid capture buffer and hybridized for 2 hr. Subsequently, DNA hybridized to the probes are separated from proteins and unbound dsDNA via magnetic beads. (FIG. 3B) NGS library preparation for sssDNA. This protocol was modified from the reported methods (Gansauge & Meyer, 2013; Snyder et al., 2016). (FIG. 3C) Bioinformatic pipeline.

FIGS. 4A-B. Sequencing results from RBC and WBC libraries. Length distribution (left) and whole genome alignment (right) of captured sssDNA from (FIG. 4A) RBC and (FIG. 4B) WBC prepared from the same healthy individual's blood. Aligned NGS reads were used for length distribution and whole genome alignment.

FIGS. 5A-E. Sequencing results from non-human RBC and WBC libraries. Length distribution (left) and whole genome alignment (right) of captured sssDNA from biospecimens from non-human species, including (FIG. 5A) monkey plasma, (FIG. 5B) plasma from mouse arterial blood, (FIG. 5C) orange juice, (FIG. 5D) peach juice, and (FIG. 5E) milk.

FIGS. 6A-D. Cross-species genome alignments. Cross-species genome alignment of sssDNAs from peach juice aligned to (FIG. 6A) peach genome and (FIG. 6B) human genome, sssDNAs from milk aligned to (FIG. 6C) cattle genome and (FIG. 6D) human genome. Aligned depth significantly dropped when aligning NGS reads to a different species.

FIGS. 7A-B. Characterization of the DCB method. (FIG. 7A) Sequence length distribution of sssDNAs captured from plasma and spike-in reference sssDNAs. Approximated concentration of sssDNA in plasma is 1.4 ng/mL. (FIG. 7B) Bar graph of NGS reads count of spike-in ssDNA1 and spike-in ssDNA2 or dsDNA2 (ssDNA2 pre-annealed to its complement strand).

FIGS. 8A-C. Direct capture from biospecimen (DCB) method for extracting sssDNA and ussDNA from blood plasma. (FIG. 8A) DCB workflow. A biotin-modified DNA capture probe bearing degenerate poly-N random sequence (NNNNNNNNNN=SEQ ID NO: 2) is directly added to plasma and hybridized for 2 hr. Subsequently, DNA hybridized to the probes are separated from proteins and unbound dsDNA via magnetic beads. Depending on whether dsDNA, such as cfDNA, from the biospecimen is also of interest, DCB includes an optional initial heat-denaturation step. (FIG. 8B) NGS library preparation for sssDNA and ussDNA. (FIG. 8C) Bioinformatic workflow.

FIGS. 9A-B. Preliminary NGS results on DNA extracted from plasma using DCB. (FIG. 9A) Results from applying DCB to blood plasma without heat denaturation. Plasma derived from a 10 mL whole blood sample from a healthy volunteer, commercially purchased from ZenBio. Plasma was separated from whole blood using a double-spin protocol to minimize leukocyte contamination. The observed ssDNA can be clearly separated into the ˜50 nt sssDNA peak and the ˜15 nt ussDNA peak. The bottom panel shows a zoom-in; very few ssDNA molecules are found in plasma with length between ˜100 nt and ˜200 nt. (FIG. 9B) Comparative results from applying DCB to blood plasma after 95° C. heat treatment for 30 minutes. The small peak at ˜166 nt is believed to be double-stranded cell-free DNA in plasma. The relative areas under the ˜50 nt sssDNA peak and the ˜166 nt cfDNA peak implies that the concentration of sssDNA in plasma is higher than that of cfDNA in plasma.

FIG. 10. Alignment of sssDNA from FIGS. 9A-B to the human genome. Over 90% of sssDNA reads between ˜35 nt and ˜50 nt mapped to the human genome.

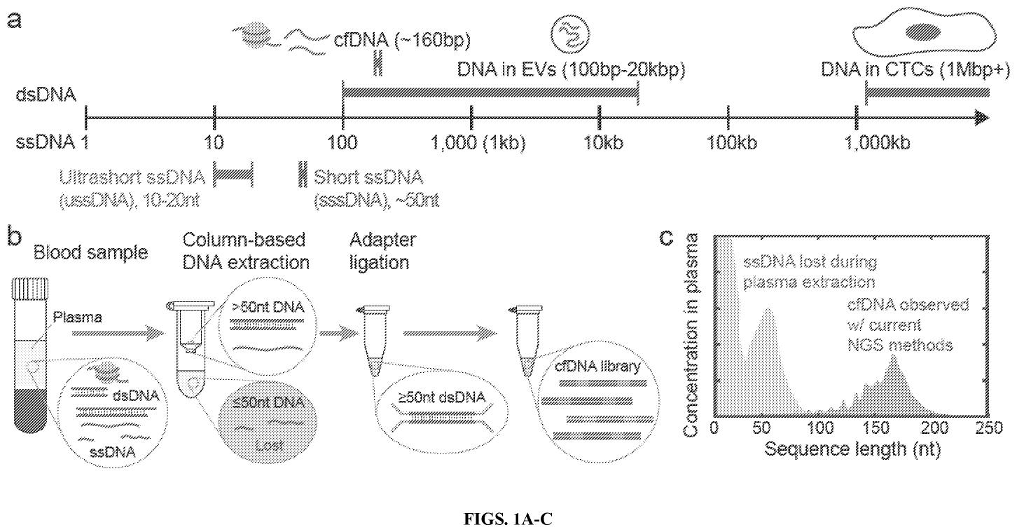

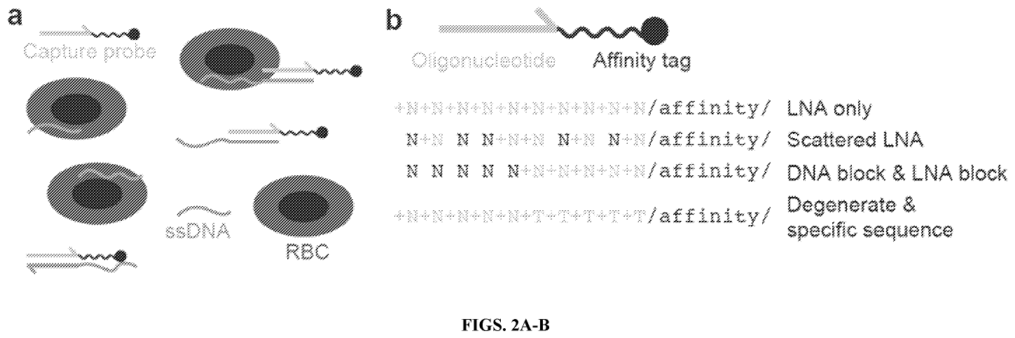

### DETAILED DESCRIPTION

Hybrid capture-based methods to extract single-stranded DNA or RNA directly from non-treated biospecimens are provided herein. These methods allow for the discovery of unexplored short single-stranded DNA (sssDNA, mean length 50 nt) and ultrashort single-stranded DNA (ussDNA, mean length 15 nt) of human origin present in plasma. The DNA or RNA extracted using the disclosed methods here can be used as disease prognostic biomarkers and treatment predictive biomarkers. For example, the DNA or RNA extracted can be sequenced to identify mutation sequence variance or quantitative relative concentrations of single or multiple DNA or RNA molecules.

Compared to previous methods to extract DNA or RNA from biospecimen, the present methods can be directly applied to non-treated biospecimens, such as plasma, serum, blood, urine, cerebrospinal fluid, and sputum. In addition, the present methods are hybrid-capture based, and thus overcome the loss of short DNA and single-stranded DNA in existing DNA extraction methods, which are based on silica-DNA interactions using columns or beads. The methods also enable the discovery of unexplored short single-stranded DNA (sssDNA, mean length 50 nt) and ultrashort single-stranded DNA (ussDNA, mean length 15 nt) of human origin present in plasma.

### I. DEFINITIONS

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 30-100 “cycles” of denaturation and replication.

“Biospecimen,” as used herein, includes, but is not limited to, plasma, serum, blood, urine, cerebrospinal fluid, tears, lymph fluid, peritoneal fluid, ascites fluid, umbilical cord blood, amniotic fluid, and sputum. In some embodiments, a biospecimen may not be subjected to various treatments, such as chemical modification and fragmenting treatments. Fragmenting treatments include mechanical, sonic, chemical, enzymatic, degradation over time, etc. Chemical modifications include bisulfite conversion and methylation/demethylation.

In certain aspects, the “capture probes” have a stretch of about 10 (e.g., 7, 8, 9, 10, 11, or 12) degenerate nucleotides. The term “degenerate” as used herein refers to a nucleotide or series of nucleotides wherein the identity can be selected from a variety of choices of nucleotides, as opposed to a defined sequence. The capture probe sequence may be NNNNNNNNNNTTTTT/3Bio/ (SEQ ID NO: 1), wherein N represents positions containing any one of multiple nucleotides. As such, the capture probe may have a 5′ degenerate region (e.g., 10 N residues) and a 3′ region having a known sequence (e.g., five T residues). A probe library with 10 variable positions, and 4 possible nucleotides at each position is comprised of 4¹⁰=1,048,576 members. In a particular embodiment, the capture probe oligonucleotides are biotin-functionalized at the 3′ end, and streptavidin-functionalized magnetic beads are added to solution after the hybridization reaction between the biospecimen and the probes. Washing the magnetic bead suspension in the vicinity of a magnet removes unbound molecules.

The term “ligase” as used herein refers to an enzyme that is capable of joining the 3′ hydroxyl terminus of one nucleic acid molecule to a 5′ phosphate terminus of a second nucleic acid molecule to form a single molecule. The ligase may be a DNA ligase or RNA ligase.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

As used herein, a nucleic acid “region” or “domain” is a consecutive stretch of nucleotides of any length.

The term “nucleic acid” or “polynucleotide” will generally refer to at least one molecule or strand of DNA, RNA, DNA-RNA chimera or a derivative or analog thereof, comprising at least one nucleobase, such as, for example, a naturally occurring purine or pyrimidine base found in DNA (e.g., adenine “A,” guanine “G,” thymine “T” and cytosine “C”) or RNA (e.g. A, G, uracil “U” and C). The term “nucleic acid” encompasses the terms “oligonucleotide” and “polynucleotide.” Note that although oligonucleotide and polynucleotide are distinct terms of art, there is no exact dividing line between them and they are used interchangeably herein. These definitions generally refer to at least one single-stranded molecule, but in specific embodiments will also encompass at least one additional strand that is partially, substantially, or fully complementary to at least one single-stranded molecule. Thus, a nucleic acid may encompass at least one double-stranded molecule. As used herein, a single stranded nucleic acid may be denoted by the prefix “ss,” and a double-stranded nucleic acid by the prefix “ds.” Notably, ssDNA is composed of nucleotides, while dsDNA is composed of base pairs, i.e., complementary nucleotide pairs. The nucleic acid molecule can be transformed from RNA into DNA and from DNA into RNA. For example, and without limitation, mRNA can be created into complementary DNA (cDNA) using reverse transcriptase and DNA can be created into RNA using RNA polymerase. A nucleic acid molecule can be of biological or synthetic origin.

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double-stranded nucleic acid molecule during hybridization. In certain embodiments, the term “substantially complementary” refers to at least one nucleic acid that may hybridize to at least one nucleic acid strand or duplex in stringent conditions. In certain embodiments, a “partially complementary” nucleic acid comprises at least one sequence that may hybridize in low stringency conditions to at least one single or double-stranded nucleic acid, or contains at least one sequence in which less than about 70% of the nucleobase sequence is capable of base-pairing with at least one single or double-stranded nucleic acid molecule during hybridization.

A “nucleoside” is a base-sugar combination, i.e., a nucleotide lacking a phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e., dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

“Nucleotide,” as used herein, is a term of art that refers to a base-sugar-phosphate combination. Nucleotides are the monomeric units of nucleic acid polymers, i.e., of DNA and RNA. The term includes ribonucleotide triphosphates, such as rATP, rCTP, rGTP, or rUTP, and deoxyribonucleotide triphosphates, such as dATP, dCTP, dUTP, dGTP, or dTTP.

“Solid support,” as used herein, means a solid carrier, including, but not limited to, a microtiter plate, beads (e.g., magnetic, glass, plastic, or metal coated beads), slides (e.g., glass or gold-coated slides), micro- or nano-particles, solid support platinum, palladium, microfluidization chamber, or channel carbon. In some cases, a solid support may be a solid support based on silicon oxide, a plastic polymer-based solid support (e.g., nylon, nitrocellulose or polyvinyl fluoride-based solid support), or a bio-based polymer (e.g., cross-linked dextran or cellulose-based solid support) solid support. A capture probe may be able to be pulled-down, directly or indirectly, using a solid support. For example, biotin can be a component of the capture probe, which can interact with a streptavidin-coated solid support.

### II. DIRECT CAPTURE OF SSSDNA FROM RED BLOOD CELLS

In one embodiment, the direct capture approach is applied to extract single-stranded DNA from different blood components, namely, plasma, red blood cell layer, and white blood cell layer. Investigating the sssDNA content in the RBC layer, which is believed to be deprived of nucleic acids, is of particular interest.

RBC layer was separated from total blood by density gradient centrifugation. Freshly drawn blood was separated by centrifuging at 1,500×g for 20 min at room temperature. The upper clear plasma layer was first removed without interrupting the interface, and the interface was gently disrupted and moved to the side by a P1000 tip. The RBC was then collected by slowly drawing from the bottom-most liquid and leaving some RBC layer with the interface to avoid white blood cell contamination.

The isolated RBCs are mixed to capture probe and hybrid capture buffer and incubate at room temperature for 2 hrs with shaking to allow hybridization of sssDNA and capture probe. The capture probe is a 10-mer with degenerated LNA bases and biotin modification (5′-+N+N+N+N+N+N+N+N+N+N/iSp18//3Bio/-3′ (SEQ ID NO: 2)). The hybrid capture reaction comprises 2 μM of capture probe, 0.5 M NaCl, 1× TE, and 0.1% Tween-20.

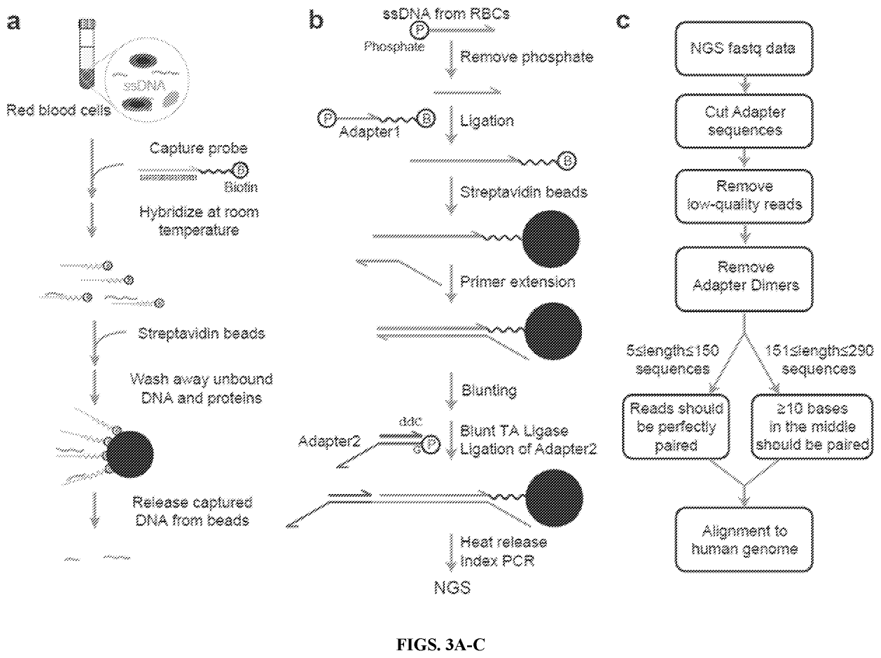

Next, MyOne C1 streptavidin beads were added to the mixture, and incubated at room temperature for 30 min. The tube containing reaction mixture was put on a magnetic rack to remove and discard supernatant, and the remaining streptavidin beads were washed with buffer containing 0.5 M NaCl, 1× TE, and 0.1% Tween-20. Captured DNA was released from streptavidin beads by heating beads at 95° C. in water (FIG. 3A).

### III. DIRECT CAPTURE FROM BIOSPECIMEN (DCB)

As described herein, methods for extracting short single-stranded DNA (ssDNA) using the direct capture from biospecimen (DCB) methods can be performed, for example, on human plasma samples. The DBC method can also be applied to biospecimens derived from non-human species, including plasma sample from monkey, plasma from mouse arterial blood, freshly prepared orange juice and peach juice, and milk. These methods provide for the detection and analysis of unexplored short single-stranded DNA (sssDNA, mean length 50 nt) and ultrashort single-stranded DNA (ussDNA, mean length 15 nt) of human origin present in plasma. The concentrations (in ng/mL) of sssDNA and ussDNA are higher than that of cfDNA (around 167 bp).

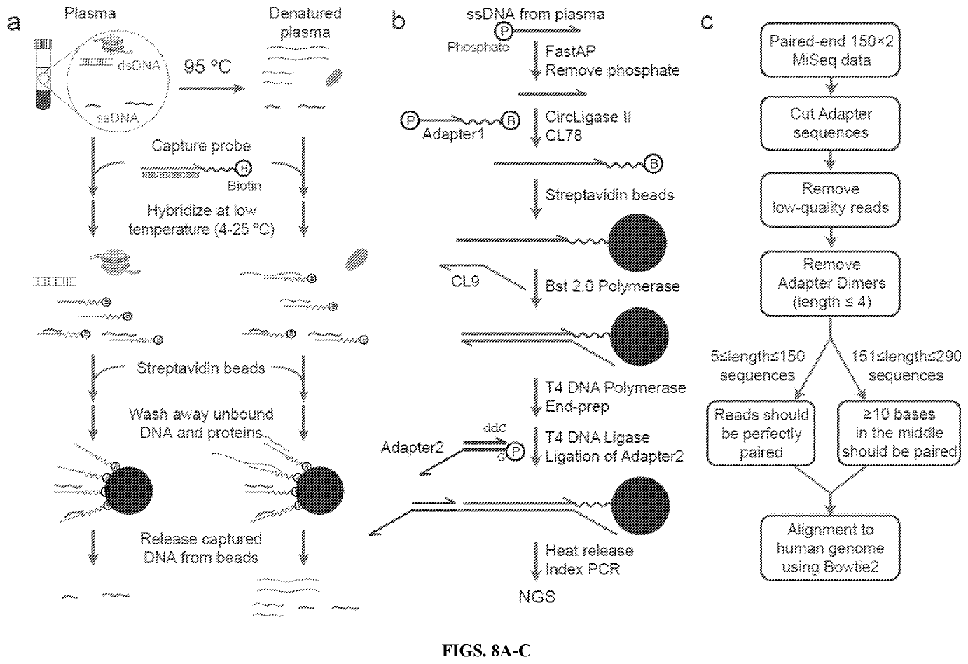

High-yield extraction of short single-stranded DNA can be achieved by the direct application of degenerate poly-N DNA probes to blood plasma to allow hybridization of short single-stranded DNA to the probes. The DCB workflow is summarized in FIG. 8A. To maximize the capture yield of all DNA molecules, especially ussDNA molecules, the DNA probes were designed to be very short (˜10 nt), and the hybridization performed at a low temperature in a high salinity buffer. This allows all ssDNA molecules at least about 10 nt long to bind with high affinity. Importantly, when performing DCB on non-treated biospecimens, double-stranded cell-free DNA and DNA encapsulated in cells or exosomes will not be extracted.

To co-extract cell-free DNA along with ssDNA, the plasma is first treated with protease and heat-denatured prior to DCB. Because the concentrations of cfDNA in the plasma are low, it is highly unlikely that denatured dsDNA rehybridizes on the timescale of the subsequent magnetic bead separation.

In one embodiment, heat-denatured plasma samples are prepared by first digesting proteins in the plasma using Protease K (56° C., 30 min), and then incubating at 98° C. for 15 min to denature the DNA and deactivate Protease K. In another embodiment, unprocessed plasma is directly used as input for DCB.

Unprocessed or heat-denatured plasma samples were then mixed with the capture probe, NaCl solution, TE buffer, and Tween-20 to result in a mixture containing 2 mM capture probe, 0.5 M NaCl, 0.8× TE, and 0.08% Tween-20. The capture probe sequence was NNNNNNNNNNTTTTT/3Bio/(SEQ ID NO: 1). The hybridization reaction was incubated at room temperature (25° C.) for 2 hrs. Next, MyOne C1 streptavidin beads were added to the mixture and incubated at room temperature for 30 min. The tube containing the reaction mixture was put on a magnetic rack to remove and discard supernatant, and the remaining streptavidin beads were washed with buffer containing 0.5 M NaCl, 1× TE, and 0.1% Tween-20. Captured DNA was released from streptavidin beads by heating beads at 95° C. in water (FIG. 8A).

### IV. SINGLE-STRANDED SEQUENCING LIBRARY PREPARATION AND SEQUENCING

In some embodiments, the captured sssDNAs are amended with Illumina sequencing adapters and sequenced on Miseq. The subsequent NGS library preparation process for sssDNA extracted from DCB or RBC utilizes the CircLigase enzyme, which acts on single-stranded DNA (FIGS. 3A & 8B). The single-stranded sequencing library preparation protocols and sequences for the oligonucleotides used in library preparation (Adapter 2, CL9, CL78) are based on the methods previously reported (Gansauge & Meyer, 2013; Snyder et al., 2016). FastAP enzyme was used to remove the 5′ phosphate from the extracted DNA, and then the biotin-modified, single-stranded CL78 was ligated using CircLigase II. The CircLigase II was used as a single-strand ligase, and the phosphate was removed to prevent circularization and polymerization of ssDNA. The ligation product was captured by streptavidin beads, and second-strand synthesis was performed on-beads using a primer CL9 and Bst 2.0 polymerase. Next, the end-prep reaction was performed using T4 DNA polymerase, and the double-stranded Adapter 2 was ligated using Blunt T4 DNA ligase. The DNA products containing both NGS adapters were then released from beads by heating to 95° C. in water; then index PCR was performed, and the resulting library was ready for NGS. All enzymes were used at near-room-temperature or below-room-temperature conditions, so that the short double strands formed in the process would not dissociate (FIGS. 3B & 8B).

The library was sequenced by Miseq. After sequencing, NGS adapter sequences were first removed from paired-end NGS reads, and low-quality reads were also removed. Reads that are too short (length≤4 nt) were removed, because they are likely adapter dimers. Non-paired reads were also removed. Sequences with lengths between 5 nt and 150 nt needed to be perfectly paired, and sequences with lengths between 151 nt and 290 nt needed to have at least 10 paired bases in the middle of the sequence (FIGS. 3C & 8C).

### V. CAPTURE PROBE DESIGN

Different capture probes were tested to improve on-target rate and reduce artifact derived from residual capture probe included in the final library. Four capture probes were tested and fraction of capture probe-derived reads were summarized in Table 1. Comparing to TTTTT as the spacer between poly N and 3′ biotin, spacer that cannot be recognized by polymerase (such as iSp3 and iSp9 from IDT) reduced artifacts from probes. The probe-derived sequences were further removed by using Locked nucleic acid (LNA) probe with a spacer that cannot be recognized by polymerase.

TABLE 1
3' spacer in probe reduces probe-derived
sequences in final library

| | | Fraction |
|---|---|---|
| | | of |
| | | capture |
| | | probe- |
| | Capture probe | derived |
| | sequence | reads |
| DNA probe | NNNNNNNNNNTTTT | 34.1% |
| with | T/3Bio/ | |
| polyT | | |
| DNA probe | NNNNNNNNNN/iSpC3// | 47.4% |
| with 3 | iSp9//iSp9//3Bio/ | |
| spacer | | |
| modifications | | |
| DNA probe | NNNNNNNNNN/iSpC3// | 6.3% |
| with 4 | iSpC3//iSp9// | |
| spacer | iSp9//3Bio/ | |
| modifications | | |
| LNA probe | +N+N+N+N+N+N+N+N+ | 0.2% |
| | N+N/iSpl8//3Bio/ | |

(NNNNNNNNNNTTTTT = SEQ ID NO: 1; NNNNNNNNNN = SEQ ID NO: 2)

### VI. KITS

The technology herein includes kits for performing the direct capture from biospecimen methods provided herein. A “kit” refers to a combination of physical elements. For example, a kit may include, for example, one or more components, such as randomer capture probes, as well as, streptavidin-coated beads, enzymes, reaction buffers, primers for NGS library preparation, an instruction sheet, and other elements useful to practice the technology described herein. These physical elements can be arranged in any way suitable for carrying out the disclosure.

The components of the kits may be packaged either in aqueous media or in lyophilized form. The container means of the kits will generally include at least one vial, test tube, flask, bottle, syringe or other container means, into which a component may be placed, and preferably, suitably aliquoted (e.g., aliquoted into the wells of a microtiter plate). Where there is more than one component in the kit, the kit also will generally contain a second, third or other additional container into which the additional components may be separately placed. However, various combinations of components may be comprised in a single vial. The kits of the present disclosure also will typically include a means for containing the nucleic acids, and any other reagent containers in close confinement for commercial sale. Such containers may include injection or blow molded plastic containers into which the desired vials are retained.

A kit will also include instructions for employing the kit components as well the use of any other reagent not included in the kit. Instructions may include variations that can be implemented. It is contemplated that such reagents are embodiments of kits of the disclosure. Such kits, however, are not limited to the particular items identified above.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### —Length Distribution and Human Genome Alignment of sssDNA Extracted from Blood Components

Total blood was separated as previously described, from which RBC layer, WBC layer (with some RBCs) from the same healthy individual were prepared for sssDNA capture and NGS library preparation. The sequencing results were analyzed for length distribution and whole genome alignment.

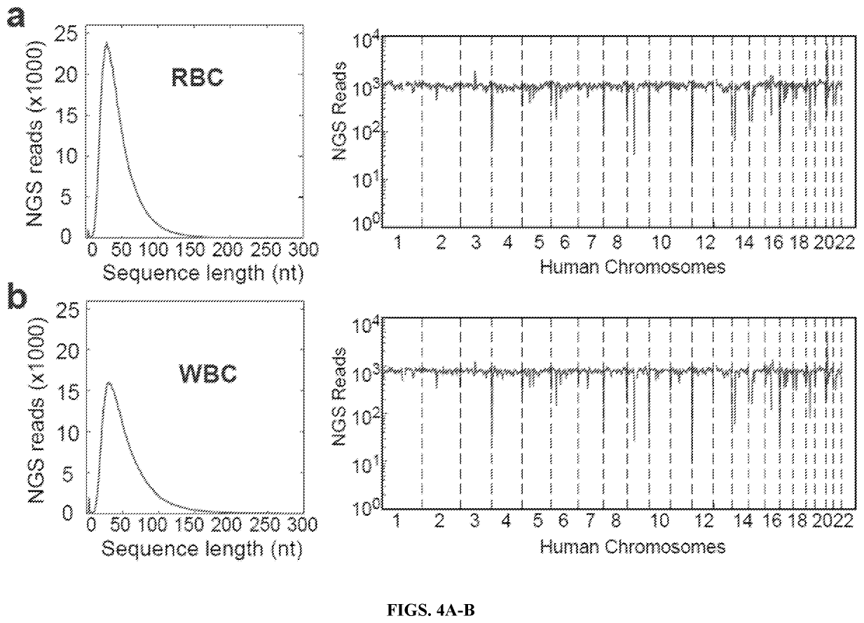

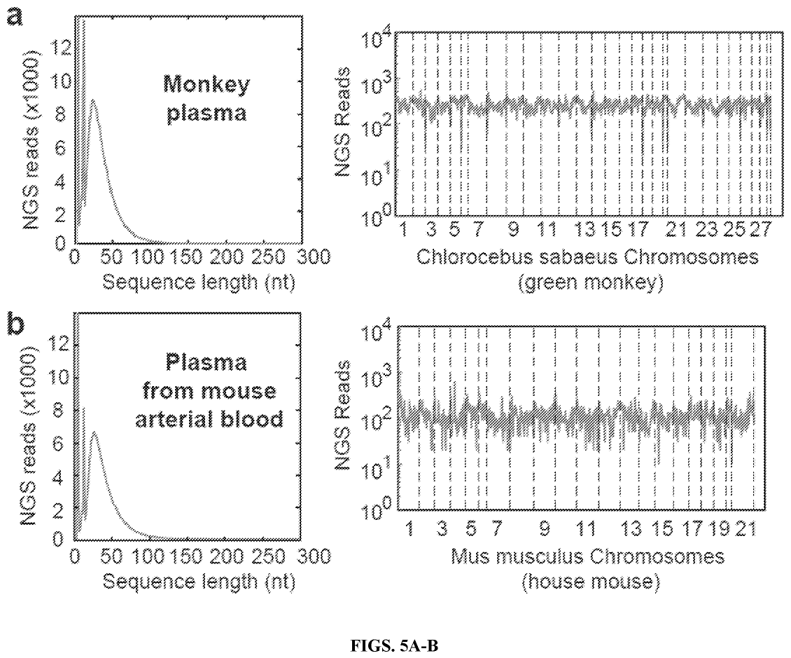

FIGS. 4A-B shows the sequencing results from RBC and WBC libraries, respectively. Extracted sssDNAs exhibited similar length distribution in RBC and WBC libraries, with the majority of sssDNA shorter than 100 nt. This potentially represent a distinct DNA species that has not been reported, since the length is pronouncedly shorter than cell-free DNA found in plasma at around 165 bp and the extracted DNAs are single-stranded or comprise a single-stranded domain. This population is likely be lost in conventional spin column- or magnetic bead-based DNA extraction methods as they present significantly lower yield at size below 50 bp. Strikingly, a large amount of short sssDNA were found in RBCs which were believed to lack DNA due to the lack of nuclei in >97% of RBCs. Based on the quantification using qPCR of sssDNA amended with Illumina index sequences, the concentrations of sssDNA were comparable in RBC and WBC layers, and more than 100× higher than that in plasma. Here, the WBC layer was mixed with RBCs because upon centrifugation, buffy coat became a thin layer and the RBCs in direct contact with the layer were collected along with WBCs. Since RBC concentration is 20× higher than that of WBC in blood, the layer of WBC is likely to have a significant fraction of RBC. These results suggest that the extracted sssDNAs are potentially associated with cell membrane, or cell membrane of RBCs only. Precise separation of different blood separations will help further decipher the origin of sssDNAs.

Next, these reads were aligned using Bowtie 2 to the human genome, and over 90% of the reads mapped to the human genome (FIG. 4). Furthermore, the mapped positions show a roughly uniform distribution across the entire human genome.

### Example 2

### Concentration of sssDNA in Human Plasma

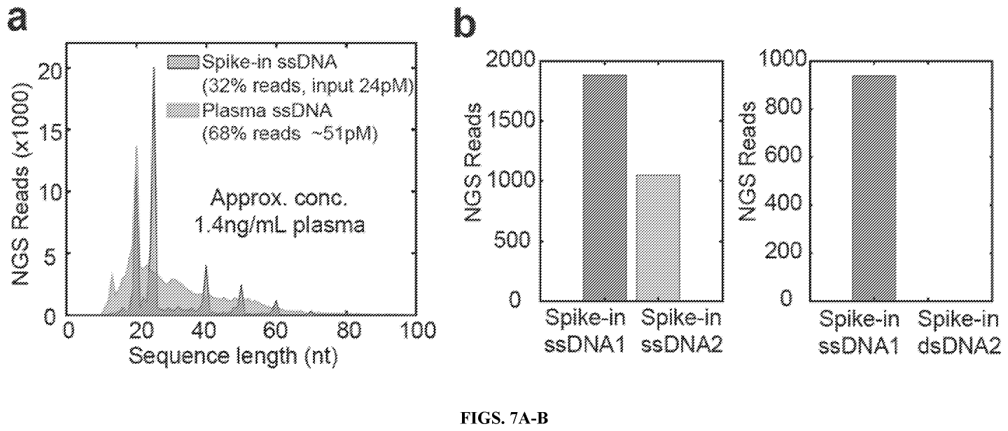

Spike-in reference DNAs were used to estimate the concentration of sssDNA in plasma from a healthy volunteer. Reference DNAs were synthetic single-stranded DNAs with length of 20 nt, 30 nt, 40 nt, 50 nt, 60 nt and 70 nt, and at each length four different sequences were added to hybrid capture solution at 1 pM per strand. The capture mixture comprised 100 μL of human plasma, and 24 pM of total spike-in DNA in a total of 240 μL mixture. FIG. 7A exhibited sequence lengths of captured molecules from plasma or spike-in reference. The sequence lengths at the spike-in sizes displayed spiky distributions. Reads longer than 10 nt were aligned to reference sequences of the spike-in strands, and the aligned spike-in reads took 32% of all aligned reads. The sssDNA concentration in plasma is estimated from the relative abundance, which comes to 51 pM. And the mass concentration is approximated from the following calculation:

(51 pM/100 μL plasma)×(240 μL)×(average size 35 nt)×(330 g/mol/nt)=1.4 ng/mL plasma.

### Example 3

### Capture Efficiency of Single-Stranded and Double-Stranded DNA

Whether the DCB method primarily captures ssDNA was tested. Two spike-in ssDNA strands were added at 1 pM to hybrid capture solution, and the NGS reads aligned to their sequences are within 2-fold. However, when spike-in ssDNA2 was pre-annealed to its complementary strand and added to the system as dsDNA, its reads became less than 1% of the other spike-in ssDNA (FIG. 7B).

### Example 4

### Direct Capture from Non-Human-Derived Biospecimens

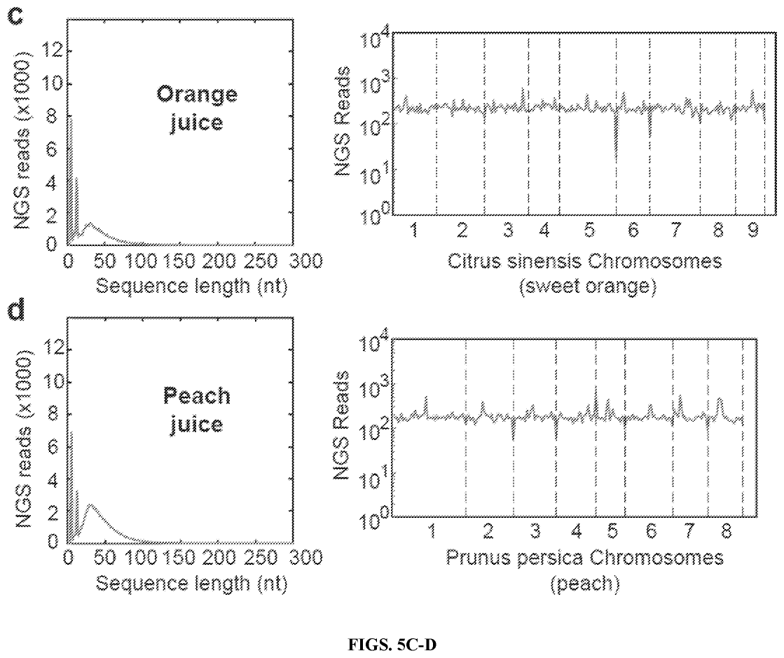

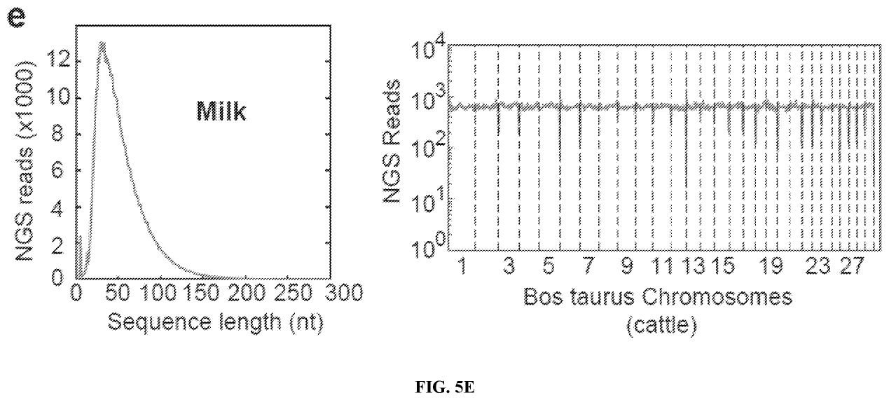

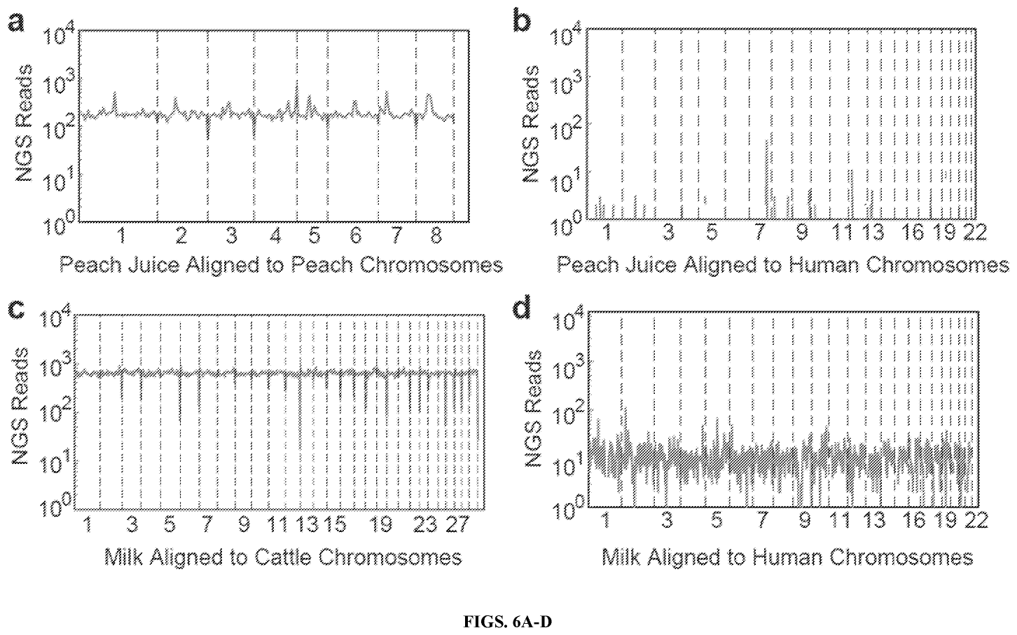

The DBC method was also applied to biospecimens derived from non-human species, including plasma sample from monkey, plasma from mouse arterial blood, freshly prepared orange juice and peach juice, and milk. Direct capture found similar distribution of short sssDNAs as seen in human specimens, and the captured sssDNAs displayed uniform distribution throughout whole genome of the corresponding species (FIGS. 5A-E). sssDNA sequences from peach juice and milk were aligned to human genome and showed scarcely aligned sequences or significantly dropped aligned depth (FIG. 6). Thus, the cross-species alignment validated that sssDNA libraries contains primarily true molecules in the corresponding biospecimens. The concentrations of sssDNAs in monkey plasma, orange juice and peach juice are speculated to be low because predominant peaks <10 nt indicating adapter dimer were observed in the NGS libraries (FIGS. 5A-D). Interestingly, milk purchased from grocery and stored at 4° C. until use was found to be enriched in sssDNA. The fact that sssDNAs are found in non-human primate, plant, and secreted biofluid may suggest universal existence of the DNA type.

### Example 5

### Length Distribution and Human Genome Alignment of ssDNA Extracted via DCB

Direct capture from biospecimen (DCB) method for extracting ssDNA has been developed and tested in both non-treated and heat-denatured healthy plasma samples. The extracted ssDNA was analyzed by single-stranded sequencing library preparation and sequencing as described above. Two classes of unexplored single-stranded DNA have been discovered in human plasma.

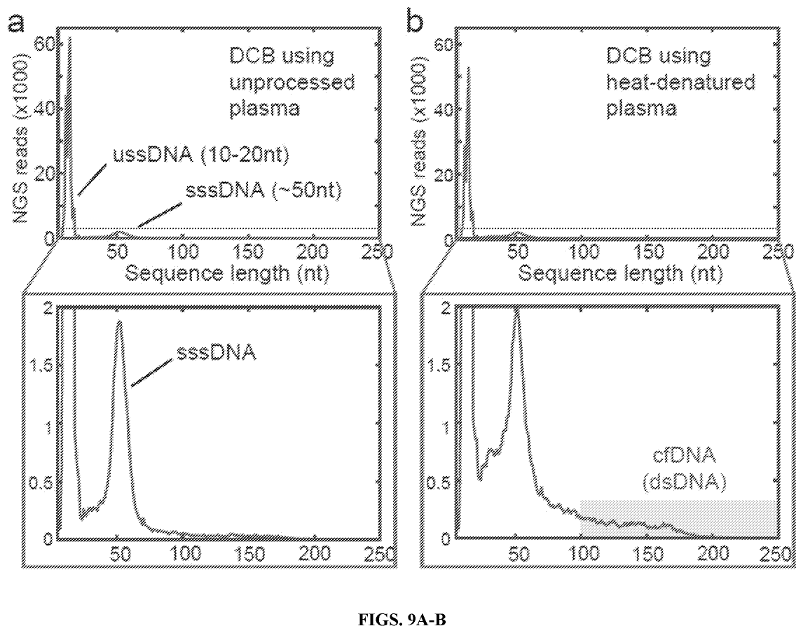

The typical NGS results for one individual's plasma (both non-treated and heat-denatured) are summarized in FIGS. 9A-B. FIG. 9A shows the results of DCB applied to plasma immediately after separation from whole blood using a double-spin protocol to minimize leukocyte contamination of plasma. The NGS results thus reflect the single-stranded DNA in plasma that were captured by DCB. There are two prominent peaks, corresponding to distinct populations of single-stranded DNA in plasma: sssDNA with a mean length of 50 nt and a tight distribution between 35 nt to 65 nt, and ussDNA with a mean length of roughly 15 nt and few molecules longer than 20 nt. The length distribution of sssDNA strongly suggests that they are a discrete set of ssDNA present in plasma. Random DNA fragmentation or PCR bias towards shorter amplicons would result in a more continuous length distribution favoring shorter DNA molecules and would not result in a relative void of ssDNA molecules between ˜20 nt and ˜35 nt long.

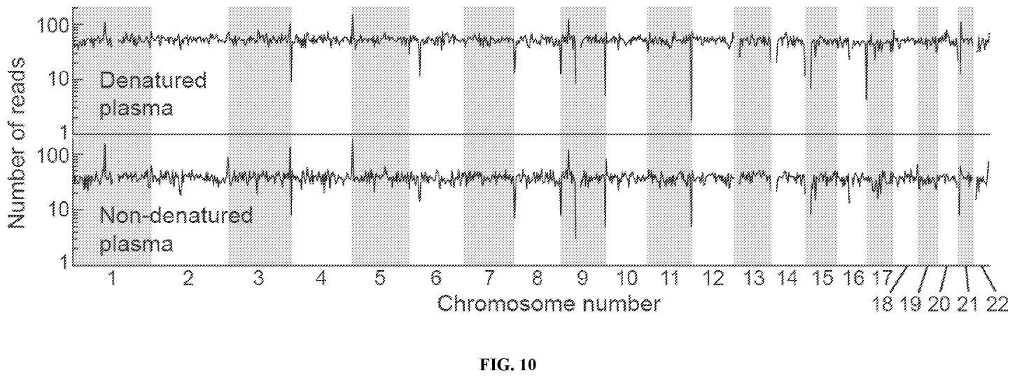

The reads were aligned to the human genome using Bowtie 2, and over 90% of the reads mapped to the human genome (FIG. 10). Furthermore, the mapped positions show a roughly uniform distribution across the entire human genome. The presence and concentrations of the sssDNA were similar in the blood plasma from two human plasma samples tested.

### Example 6

### Concentration of ssDNA Extracted via DCB

The concentration of ussDNA appears to be far higher than that of sssDNA based on the NGS sequencing results. Due to the short length of ussDNA (˜15 nt), the sequences map nonspecifically to the genomes of many different species, so it is difficult to verify if any given ussDNA molecule has a human origin. However, there is a high diversity of different ussDNA sequences, so it is likely that many ussDNA are of human origin.

The concentration of sssDNA was quantified through comparison to cfDNA by applying DCB after heat-denaturation of plasma (FIG. 9B). This process denatures cfDNA and renders it single-stranded, so it can be captured and represented in the NGS library. The length distribution of ssDNA molecules in denatured plasma samples shows a small but significant peak at roughly ˜166 nt (FIG. 9B), corresponding to the cfDNA. Even after adjusting for the 3-fold length difference between sssDNA and cfDNA, the nanograms/mL of sssDNA appears to be significantly higher than that of cfDNA. The relative concentration of ussDNA is even higher than that of sssDNA, though as previously mentioned, it is not currently possible to determine the human-derived nature of any given ussDNA.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Gansauge & Meyer, (2013). Single-stranded DNA library preparation for the sequencing of ancient or damaged DNA. Nature protocols, 8(4), 737.
Snyder et al., (2016). Cell-free DNA comprises an in vivo nucleosome footprint that informs its tissues-of-origin. Cell, 164(1-2), 57-68.

What is claimed is:

1. A method for capturing short single-stranded DNA (sssDNA) from red blood cells, the method comprising:
(a) isolating red blood cells from freshly drawn blood;
(b) contacting the isolated red blood cells with a capture probe comprising an oligonucleotide having a length between 5 nt and 100 nt and an affinity tag;
(c) incubating the isolated red blood cells with the capture probe at a temperature between 0° C. and 45° C. for between 1 second and 1 day to allow for hybridization between the capture probe and sssDNA;
(d) collecting the capture probes using the affinity tag; and
(e) washing the collected capture probes and collecting captured DNA in an elution buffer.

2. The method of claim 1, wherein the freshly drawn blood is collected in anticoagulant coated tubes.

3. The method of claim 1, wherein red blood cells are isolated by density gradient centrifugation, fluorescence-activated cell sorting (FACS), or white blood cells depeletion using immunomagnetic cell separation.

4. The method of claim 1, wherein the biospecimen has not been stored at a temperature above 4° C. for more than 48 hours prior to performing the method.

5. The method of claim 1, wherein the red blood cells are not isolated from blood that has undergone a freeze-thaw cycle.

6. The method of claim 1, wherein the red blood cells have not been heated above 45° C.

7. The method of claim 1, wherein the red blood cells have not undergone any enzymatic reactions prior to performing the method.

8. The method of claim 7, wherein the red blood cells have not been treated with proteinase K prior to performing the method.

9. The method of claim 1, wherein the red blood cells have not undergone any chemical treatments prior to performing the method.

10. The method of claim 9, wherein the red blood cells have not been lysed prior to performing the method.

11. The method of claim 1, wherein the red blood cells have not undergone any harsh physical treatments prior to performing the method.

12. The method of claim 11, wherein the red blood cells have not been sheared prior to performing the method.

13. The method of claim 11, wherein the red blood cells have not been electroporated prior to performing the method.

14. The method of claim 11, wherein the red blood cells have not been sonicated prior to performing the method.

15. The method of claim 1, wherein the affinity tag is a noncovalent affinity tag.

16. The method of claim 15, wherein the affinity tag is biotin.

17. The method of claim 1, wherein the affinity tag is a covalent affinity tag.

18. The method of claim 17, wherein the affinity tag is an azide or alkyne functional group.

19. The method of claim 1, wherein the oligonucleotide of the capture probe comprises a region of unmodified degenerate bases.

20. The method of claim 19, wherein the region of unmodified degenerate bases comprises between 5 and 100 nucleotides.

21. The method of claim 19, wherein each degenerate base position can be any one of A, G, Tor C.

22. The method of claim 1, wherein the oligonucleotide of the capture probe is a DNA oligonucleotide.

23. The method of claim 1, wherein the oligonucleotide of the capture probe comprises one or more non-natural degenerate bases with non-natural backbone modifications.

24. The method of claim 23, wherein the oligonucleotide of the capture probe comprises locked nucleic acids.

25. The method of claim 1, wherein the oligonucleotide of the capture probe comprises one or more non-natural degenerate bases with universal affinity.

26. The method of claim 25, wherein the non-natural degenerate bases with universal affinity are inosine or 5-nitroindole.

27. The method of claim 1, wherein the concentration of the capture probe is between 50 pM and 5 μM.

28. The method of claim 1, wherein step (b) further comprises contacting the biospecimen with a hybrid capture buffer, wherein the hybrid capture buffer comprises 100 mM to 1 M sodium chloride, 0.01% (v/v) to 1% (v/v) Tween20, 1 mM to 100 mM Tris, 1 mM to 100 mM ethylenediaminetetraacetic acid (EDTA), 0.01% (v/v) to 1% (v/v) sodium dodecyl sulfate (SDS), and 0 M to 3 M tetramethylammonium chloride (TMAC).

29. The method of claim 1, comprising treating the biospecimen with an RNase.

30. The method of claim 1, further comprising using ligation and/or PCR to append terminal sequences on the 5′ and/or 3′ ends of the captured single-stranded nucleic acid molecules.

31. The method of claim 30, wherein the terminal sequences are adapter and index sequences for high-throughput sequencing.

32. The method of claim 30, further comprising amplifying the index-appended single-stranded molecules using index primers.

33. The method of claim 30, further comprising performing high-throughput sequencing.

34. The method of claim 33, wherein the high-throughput sequencing is performed via sequencing-by-synthesis.

35. The method of claim 33, wherein the high-throughput sequencing is performed via sequence-specific current measurements in conjunction with nanopores.

36. A method for diagnosing disease in or selecting a treatment for a patient by analyzing mutation sequence variance in sssDNA isolated from the patient.

37. The method of claim 36, wherein the analysis comprises the method of any one of claims 1-35.

38. The method of claim 36, wherein the sssDNA isolated from the patient is isolated from red blood cells.

39. The method of claim 36, wherein the sssDNA is prepared for methylation analysis.

40. The method of claim 39, wherein the sssDNA is treated with bisulfite conversion reagents to transform all unmethylated cytosine to uracil prior to library preparation for high-throughput sequencing.

41. The method of claim 39, wherein the sssDNA is treated with oxidation reagents and APOBEC to transform all unmethylated cytosine to uracil prior to library preparation for high-throughput sequencing.

42. The method of claim 36, wherein the lengths of sssDNAs are analyzed from high-throughput sequencing data, and if the sssDNAs are longer than the sequencing read lengths, then their lengths are inferred from aligned genomic positions of paired-end reads.

43. The method of claim 36, wherein genetic alterations, single nucleotide variations, deletions, insertions, translocations, and inversions are analyzed to evaluate their association with disease and disease status.

44. The method of claim 36, wherein epigenetic alterations and methylation patterns are analyzed to evaluate their association with disease and disease status.

45. The method of claim 36, wherein expression profiles, point mutations, fusion mutations, and expression levels are analyzed to evaluate their association with disease and disease status.

46. A method for diagnosing disease in or selecting a treatment for a patient by analyzing quantitative relative concentrations of different genomic loci in the sssDNA isolated from the patient.

47. The method of claim 46, wherein the analysis comprises the method of any one of claims 1-35.

48. The method of claim 46, wherein the sssDNA isolated from the patient is isolated from red blood cells.

49. The method of claim 46, wherein the lengths of sssDNAs are analyzed from high-throughput sequencing data, and if the sssDNAs are longer than the sequencing read lengths, then their lengths are inferred from aligned genomic positions of paired-end reads.

50. The method of claim 46, wherein the total concentrations of sssDNAs in a biospecimen from the patient or in different compartments of biospecimens are estimated via spiking-in of synthetic reference sssDNA strands.

51. The method of claim 46, wherein sssDNAs aligned to different genomic loci are normalized to those aligned to reference loci to estimate relative concentrations at different genomic loci.

52. The method of claim 51, wherein the genomic loci of interest include promoter regions, 5′ UTRs, 3′ UTRs, oncogenes, tumor suppressor genes, genes regulating immune responses or neurological activities.

53. The method of claim 46, wherein metagenomics of sssDNAs is analyzed for DNA concentration of different bacterial populations.

54. The method of claim 46, wherein captured sssDNAs are analyzed for aneuploidy related to non-invasive prenatal testing (NIPT) or cancer copy number variation.

55. A composition comprising (a) isolated red blood cells, wherein the isolated red blood cells comprise no more than 1 part in 1000 white blood cells; and (b) an oligonucleotide capture probe having a length between 5 nt and 100 nt and comprising degenerate locked nucleic acid nucleotides and an affinity tag modification at the 3′ end, wherein the composition does not comprise a reverse transcriptase.

56. The composition of claim 55, wherein the isolated red blood cells are isolated from venous blood of human or non-human animals.

57. The composition of claim 55, wherein the isolated red blood cells are isolated from arterial blood of human or non-human animals.

58. The composition of claim 55, wherein the isolated red blood cells have not undergone any enzymatic reactions.

59. The composition of claim 58, wherein the isolated red blood cells have not been treated with proteinase K.

60. The composition of claim 55, wherein the isolated red blood cells have not undergone any harsh chemical treatments.

61. The composition of claim 60, wherein the isolated red blood cells have not been lysed.

62. The composition of claim 55, wherein the isolated red blood cells have not undergone any harsh physical treatments.

63. The composition of claim 62, wherein the isolated red blood cells have not been sheared.

64. The composition of claim 62, wherein the isolated red blood cells have not been electroporated.

65. The composition of claim 62, wherein the isolated red blood cells have not been sonicated.

66. The composition of claim 55, wherein the isolated red blood cells have not been stored at a temperature above 4° C. for more than 48 hours.

67. The composition of claim 55, wherein the isolated red blood cells have not been heated above 45° C.

68. The composition of claim 55, wherein the affinity tag is a noncovalent affinity tag.

69. The composition of claim 68, wherein the affinity tag is biotin.

70. The composition of claim 55, wherein the affinity tag is a covalent affinity tag.

71. The composition of claim 70, wherein the affinity tag is an azide or alkyne functional group.

72. The composition of claim 55, wherein the oligonucleotide of the capture probe comprises one or more non-natural degenerate bases with non-natural backbone modifications.

73. The composition of claim 72, wherein the oligonucleotide of the capture probe comprises locked nucleic acids.

74. The composition of claim 55, wherein the oligonucleotide of the capture probe comprises one or more non-natural degenerate bases with universal affinity.

75. The composition of claim 74, wherein the non-natural degenerate bases with universal affinity are inosine or 5-nitroindole.

76. The composition of claim 55, further comprising a hybrid capture buffer, wherein the hybrid capture buffer comprises 1 mM cations, 0.01% (v/v) to 1% (v/v) Tween20, 1 mM to 100 mM Tris, 1 mM to 100 mM ethylenediaminetetraacetic acid (EDTA), 0.01% (v/v) to 1% (v/v) sodium dodecyl sulfate (SDS), and 0 M to 3 M tetramethylammonium chloride (TMAC).

77. A method for direct capture and extraction of single-stranded DNA (ssDNA) from a biospecimen, the method comprising:
(a) incubating a non-treated biospecimen with a DNA probe comprising an affinity tag and an oligonucleotide at a temperature between 0° C. and 45° C. in a solution comprising between 0.05 molar and 6 molar monovalent cations, or comprising between 0.001 molar and 2 molar divalent cations, or comprising both between 0.05 molar and 6 molar monovalent cations and between 0.001 molar and 2 molar divalent cations, for between 1 second and 1 day to allow for hybridization between the DNA probe and ssDNA in the biospecimen;
(b) collecting the DNA probes using the affinity tag; and
(c) washing the collected DNA probes to remove any non-hybridized contaminates from the biospecimen.

78. The method of claim 77, wherein at least a portion of the ssDNA is less than 50 nucleotides long.

79. The method of claim 77, wherein at least a portion of the ssDNA is less than 20 nucleotides long.

80. The method of claim 77, wherein the DNA probe in step (a) is not conjugated to a solid support.

81. The method of claim 77, wherein the method is performed without an anion exchange medium.

82. The method of claim 77, wherein the hybridization in step (a) is direct hybridization between the DNA probe and ssDNA in the biospecimen.

83. The method of claim 77, wherein the non-treated biospecimen has not been heated above 45° C. prior to performing the method.

84. The method of claim 77, wherein the non-treated biospecimen has not undergone any biological treatments prior to performing the method.

85. The method of claim 84, wherein the non-treated biospecimen has not undergone any enzymatic reactions prior to performing the method.

86. The method of claim 85, wherein the non-treated biospecimen has not been treated with proteinase K prior to performing the method.

87. The method of claim 77, wherein the non-treated biospecimen has not undergone any chemical treatments prior to performing the method.

88. The method of claim 77, wherein the non-treated biospecimen has not undergone any harsh physical treatments prior to performing the method.

89. The method of claim 88, wherein the non-treated biospecimen has not been sheared prior to performing the method.

90. The method of claim 88, wherein the non-treated biospecimen has not been electroporated prior to performing the method.

91. The method of claim 88, wherein the non-treated biospecimen has not been sonicated prior to performing the method.

92. The method of claim 77, wherein the biospecimen is selected from the group consisting of plasma, serum, blood, urine, cerebrospinal fluid, and sputum.

93. The method of claim 77, wherein the affinity tag is a noncovalent affinity tag.

94. The method of claim 93, wherein the affinity tag is biotin.

95. The method of claim 94, wherein step (b) is performed via streptavidin-coated magnetic beads and collecting is performed using a magnet.

96. The method of claim 94, wherein step (b) is performed via streptavidin-coated agarose beads and collecting is performed using centrifugal force.

97. The method of claim 77, wherein the affinity tag is a covalent affinity tag.

98. The method of claim 97, wherein the affinity tag is an azide or alkyne functional group.

99. The method of claim 77, wherein the oligonucleotide comprises a region of degenerate bases.

100. The method of claim 99, wherein the region of degenerate bases comprises between 5 and 30 degenerate bases.

101. The method of claim 99, wherein each degenerate base position can be any one of A, G, Tor C.

102. The method of claim 99, wherein the region of degenerate bases is located at the 5′ end of the oligonucleotide.

103. The method of claim 99, wherein the oligonucleotide further comprises a region of known bases.

104. The method of claim 103, wherein the region of known bases comprises about 5 thymidines.

105. The method of claim 103, wherein the region of known bases is located between the region of degenerate bases and the affinity tag.

106. The method of claim 77, further comprising (d) eluting the hybridized ssDNA from the DNA probe.

107. The method of claim 106, further comprising (e) preparing an NGS library using the eluted ssDNA.

108. The method of claim 107, wherein the extracted ssDNA is not amplified in a sequence-specific manner.

109. The method of claim 107, further comprising (f) performing NGS on the NGS library.

110. The method of claim 77, wherein the biospecimen is a human biospecimen, and wherein the extracted ssDNA is human.

111. The method of claim 77, wherein the method is a method of selectively isolating ssDNA.

112. The method of claim 109, further comprising (g) analyzing the sequences of the ssDNA to predict disease in or select a treatment for a patient from whom the biospecimen was obtained.

113. The method of claim 109, further comprising (g) analyzing the relative concentrations of the ssDNA derived from various genomic loci to predict disease in or select a treatment for a patient from whom the biospecimen was obtained.

114. A method for direct capture and extraction of single-stranded DNA (ssDNA) and double-stranded DNA (dsDNA) from a biospecimen, the method comprising:
(a) heating the biospecimen at a minimum of 90° C. for a minimum of 10 seconds;
(b) contacting the biospecimen with a capture probe comprising an oligonucleotide having a length between 5 nt and 100 nt and an affinity tag that allows for strong association with a solid-state substance;
(c) incubating the biospecimen with the capture probe at a temperature between 0° C. and 45° C. for between 1 second and 1 day to allow for hybridization between the capture probe and nucleic acids in the biospecimen;
(d) collecting the capture probes using the affinity tag; and
(e) washing the collected capture probes and collecting captured nucleic acid.

115. The method of claim 114, wherein the biospecimen comprises isolated red blood cells, isolated platelets, isolated white blood cells, blood, plasma, serum, urine, cerebrospinal fluid, and/or sputum.

116. The method of claim 114, wherein the biospecimen is from a human, an animal, a plant, or a bacterium.

117. The method of claim 114, wherein the biospecimen is a human biospecimen, and wherein the extracted ssDNA is human.

118. The method of claim 114, wherein the biospecimen is a human microbiome specimen.

119. The method of claim 118, wherein the human microbiome specimen is an oral, a skin, a vaginal, or a fecal biospecimen.

120. The method of claim 114, wherein the biospecimen has not undergone any biological treatments prior to performing the method.

121. The method of claim 120, wherein the biospecimen has not undergone any enzymatic reactions prior to performing the method.

122. The method of claim 121, wherein the biospecimen has not been treated with proteinase K prior to performing the method.

123. The method of claim 114, wherein the biospecimen has not undergone any chemical treatments prior to performing the method.

124. The method of claim 123, wherein the biospecimen has not been lysed prior to performing the method.

125. The method of claim 114, wherein the biospecimen has not undergone any harsh physical treatments prior to performing the method.

126. The method of claim 125, wherein the biospecimen has not been sheared prior to performing the method.

127. The method of claim 125, wherein the biospecimen has not been electroporated prior to performing the method.

128. The method of claim 125, wherein the biospecimen has not been sonicated prior to performing the method.

129. The method of claim 114, wherein the biospecimen has not been stored at a temperature above 4° C. for more than 48 hours prior to performing the method.

130. The method of claim 114, wherein the affinity tag is a noncovalent affinity tag.

131. The method of claim 130, wherein the affinity tag is biotin.

132. The method of claim 131, wherein step (d) is performed via streptavidin-coated magnetic beads and collecting is performed using a magnet.

133. The method of claim 131, wherein step (d) is performed via streptavidin-coated agarose beads and collecting is performed using centrifugal force.

134. The method of claim 114, wherein the affinity tag is a covalent affinity tag.

135. The method of claim 134, wherein the affinity tag is an azide or alkyne functional group.

136. The method of claim 114, wherein the oligonucleotide of the capture probe comprises a region of unmodified degenerate bases.

137. The method of claim 136, wherein the region of unmodified degenerate bases comprises between 5 and 100 nucleotides.

138. The method of claim 136, wherein each degenerate base position can be any one of A, G, Tor C.

139. The method of claim 136, wherein the region of unmodified degenerate bases is located at the 5′ end of the oligonucleotide.

140. The method of claim 136, wherein the oligonucleotide further comprises a region of known bases.

141. The method of claim 140, wherein the region of known bases comprises about 5 thymidines.

142. The method of claim 140, wherein the region of known bases is located between the region of degenerate bases and the affinity tag.

143. The method of claim 114, wherein the oligonucleotide of the capture probe is a DNA oligonucleotide.

144. The method of claim 114, wherein the oligonucleotide of the capture probe comprises one or more non-natural degenerate bases with non-natural backbone modifications.

145. The method of claim 144, wherein the oligonucleotide of the capture probe comprises locked nucleic acids.

146. The method of claim 114, wherein the oligonucleotide of the capture probe comprises one or more non-natural degenerate bases with universal affinity.

147. The method of claim 146, wherein the non-natural degenerate bases with universal affinity are inosine or 5-nitroindole.

148. The method of claim 114, wherein the concentration of the capture probe is between 50 pM and 5 μM.

149. The method of claim 114, wherein step (b) further comprises contacting the biospecimen with a hybrid capture buffer, wherein the hybrid capture buffer comprises 100 mM to 1 M sodium chloride, 0.01% (v/v) to 1% (v/v) Tween20, 1 mM to 100 mM Tris, 1 mM to 100 mM ethylenediaminetetraacetic acid (EDTA), 0.01% (v/v) to 1% (v/v) sodium dodecyl sulfate (SDS), and 0 M to 3 M tetramethylammonium chloride (TMAC).

150. The method of claim 149, wherein the hybrid capture buffer comprises between 0.05 molar and 6 molar monovalent cations, or between 0.001 molar and 2 molar divalent cations, or both between 0.05 molar and 6 molar monovalent cations and between 0.001 molar and 2 molar divalent cations.

151. The method of claim 114, comprising treating the biospecimen with an RNase.

152. The method of claim 114, comprising treating the biospecimen with a protease prior to step (a).

153. The method of claim 114, wherein the capture probe in step (a) is not conjugated to a solid support.

154. The method of claim 114, wherein the method is performed without an anion exchange medium.

155. The method of claim 114, wherein the hybridization in step (c) is direct hybridization between the capture probe and nucleic acids in the biospecimen.

156. The method of claim 114, further comprising (f) eluting the captured nucleic acids from the capture probe.

157. The method of claim 156, further comprising using ligation and/or PCR to append terminal sequences on the 5′ and/or 3′ ends of the captured single-stranded nucleic acid molecules.

158. The method of claim 157, wherein the terminal sequences are adapter and index sequences for high-throughput sequencing.

159. The method of claim 157, further comprising amplifying the index-appended single-stranded molecules using index primers.

160. The method of claim 156, further comprising (g) preparing an NGS library using the eluted nucleic acids.

161. The method of claim 160, wherein the extracted nucleic acids are not amplified in a sequence-specific manner.

162. The method of claim 160, further comprising (h) performing high-throughput sequences on the NGS library.

163. The method of claim 162, wherein the high-throughput sequencing is performed via sequencing-by-synthesis.

164. The method of claim 162, wherein the high-throughput sequencing is performed via sequence-specific current measurements in conjunction with nanopores.

165. The method of claim 162, further comprising (i) analyzing the sequences of the extracted nucleic acids to predict disease in or select a treatment for a patient from whom the biospecimen was obtained.

166. The method of claim 162, further comprising (i) analyzing the relative concentrations of the extracted nucleic acids derived from various genomic loci to predict disease in or select a treatment for a patient from whom the biospecimen was obtained.

167. A method for direct capture and extraction of RNA from a biospecimen, the method comprising:
(a) incubating a non-treated biospecimen with an RNase inhibitor and a DNA probe comprising an affinity tag and an oligonucleotide at a temperature between 0° C. and 45° C. in a solution comprising between 0.05 molar and 6 molar monovalent cations, or comprising between 0.001 molar and 2 molar divalent cations, or comprising both between 0.05 molar and 6 molar monovalent cations and between 0.001 molar and 2 molar divalent cations, for between 1 second and 1 day to allow for hybridization between the DNA probe and RNA in the biospecimen;
(b) collecting the DNA probes using the affinity tag; and
(c) washing the collected DNA probes to remove any non-hybridized contaminates from the biospecimen.

168. The method of claim 167, wherein the DNA probe in step (a) is not conjugated to a solid support.

169. The method of claim 167, wherein the method is performed without an anion exchange medium.

170. The method of claim 167, wherein the hybridization in step (a) is direct hybridization between the DNA probe and RNA in the biospecimen.

171. The method of claim 167, wherein the non-treated biospecimen has not been heated above 45° C. prior to performing the method.

172. The method of claim 167, wherein the non-treated biospecimen has not undergone any biological treatments prior to performing the method.

173. The method of claim 172, wherein the non-treated biospecimen has not undergone any enzymatic reactions prior to performing the method.

174. The method of claim 173, wherein the non-treated biospecimen has not been treated with proteinase K prior to performing the method.

175. The method of claim 167, wherein the non-treated biospecimen has not undergone any chemical treatments prior to performing the method.

176. The method of claim 167, wherein the non-treated biospecimen has not undergone any harsh physical treatments prior to performing the method.

177. The method of claim 176, wherein the non-treated biospecimen has not been sheared prior to performing the method.

178. The method of claim 176, wherein the non-treated biospecimen has not been electroporated prior to performing the method.

179. The method of claim 176, wherein the non-treated biospecimen has not been sonicated prior to performing the method.

180. The method of claim 167, wherein the biospecimen is selected from the group consisting of plasma, serum, blood, urine, cerebrospinal fluid, and sputum.

181. The method of claim 167, wherein the affinity tag is a noncovalent affinity tag.

182. The method of claim 181, wherein the affinity tag is biotin.

183. The method of claim 182, wherein step (b) is performed via streptavidin-coated magnetic beads and collecting is performed using a magnet.

184. The method of claim 182, wherein step (b) is performed via streptavidin-coated agarose beads and collecting is performed using centrifugal force.

185. The method of claim 167, wherein the affinity tag is a covalent affinity tag.

186. The method of claim 185, wherein the affinity tag is an azide or alkyne functional group.

187. The method of claim 167, wherein the oligonucleotide comprises a region of degenerate bases.

188. The method of claim 187, wherein the region of degenerate bases comprises between 5 and 30 degenerate bases.

189. The method of claim 187, wherein each degenerate base position can be any one of A, G, Tor C.

190. The method of claim 187, wherein the region of degenerate bases is located at the 5′ end of the oligonucleotide.

191. The method of claim 187, wherein the oligonucleotide further comprises a region of known bases.

192. The method of claim 191, wherein the region of known bases comprises about 5 thymidines.

193. The method of claim 191, wherein the region of known bases is located between the region of degenerate bases and the affinity tag.

194. The method of claim 167, further comprising (d) eluting the hybridized RNA from the DNA probe.

195. The method of claim 194, further comprising (e) preparing an NGS library using the eluted RNA.

196. The method of claim 195, wherein the extracted RNA is not amplified in a sequence-specific manner.

197. The method of claim 195, further comprising (f) performing NGS on the NGS library.

198. The method of claim 167, wherein the biospecimen is a human biospecimen, and wherein the extracted RNA is human.

199. The method of claim 197, further comprising (g) analyzing the sequences of the extracted RNA to predict disease in or select a treatment for a patient from whom the biospecimen was obtained.

200. The method of claim 197, further comprising (g) analyzing the relative concentrations of the extracted RNA derived from various genomic loci to predict disease in or select a treatment for a patient from whom the biospecimen was obtained.
